(19) United States
(12) Patent Application Publication
(10) Pub. No.: US 2005/0069975 A1
(43) Pub. Date: Mar. 31, 2005

(54) Methods of enhancing yields of soluble proteins

(76) Inventors: Shane Atwell, San Diego, CA (US); Ingeborg Feil, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/617,059

(22) Filed: Jul. 9, 2003

Related U.S. Application Data
(60) Provisional application No. 60/397,068, filed on Jul. 18, 2002.

(51) Int. Cl. C12P 21/06; C07H 21/04; C12N 9/16; C12N 1/21; C07K 14/47

(52) U.S. Cl. 435/68.1; 435/69.1; 435/252.33; 435/196; 435/320.1; 530/352; 536/23.2

(57) ABSTRACT

Methods are provided for increasing yields of soluble protein, including for increasing yields of soluble recombinant protein. Specifically, methods are provided for increasing the solubility and stability of recombinant proteins by co-expression of a phosphatase.

The present invention claims the benefit under 35 U.S.C. §119(e) of prior U.S. Provisional Patent Application Ser. No. 60/397,068, filed on Jul. 18, 2002.

The invention relates to methods of increasing the yields of soluble recombinantly expressed peptides and proteins. Specifically, the invention relates to increasing the solubility and stability of recombinant proteins by co-expression of a phosphatase.

Recombinant peptide and protein expression methods are essential for the subsequent analysis and use of these peptides and proteins. Companies engaged in drug discovery especially need access to large amounts of soluble protein for the identification of candidate molecules for drug development and for the optimization of these candidates into marketable drugs. In addition, access to large amounts of purified protein is needed for protein functional assay development, protein structure-function analysis, and protein structure determination. Methods of increasing recombinant protein solubility are also useful for industrial process bioreactors.

In order to obtain large amounts of protein, researchers are often required to grow excessive volumes of host cells that express a heterologous gene coding for the protein. Such cells include, for example, bacterial, fungal, insect, and mammalian cells. In some cases, especially those that involve the use of mammalian cells as hosts, costs become prohibitive. Even large volume over-production of a protein does not always yield adequate amounts of soluble protein. During high-level, heterologous expression, proteins often aggregate in the host cell, thereby forming insoluble protein aggregate complexes commonly referred to as “inclusion bodies.” This often occurs, for example, when a eukaryotic protein is expressed in a prokaryotic system. Even in those rare cases that an insoluble protein can be denatured in urea and refolded in vitro, full activity is not always restored.

Therefore, there exists in the art a need for a general method for improving recombinant protein solubility.

### SUMMARY OF THE INVENTION

The present invention relates to the unexpected observation that co-expression of a phosphatase with a targeted, selected, or chosen protein or peptide of interest correlates with improved protein solubility, stability, and yields during subsequent purification.

In one embodiment, the present invention provides a method of improving the folding, solubility, and stability of a target protein produced in a host cell comprising co-expressing the target protein with a phosphatase. The target protein may, for example, be expressed from a nucleic acid sequence that is native to the host cell, or may be expressed from a nucleic acid sequence that is heterologous to the host cell. By “co-expression” is meant that the target, selected, or chosen protein, or peptide, of interest, is expressed in an expression system, including, for example, in a cell, or in vivo, contemporaneously with a phosphatase, so that both proteins are present in the expression system at the same time. Co-expression also includes expressing the targeted protein or peptide of interest in a host cell where the expression of an endogenous phosphatase is increased, for example, by co-transfecting a nucleotide sequence encoding a promoter that increases transcription of the endogenous phosphatase. By contemporaneous expression is meant that expression may occur simultaneously, where both target and phosphatase molecules are synthesized at the same time, or in series, where one of the proteins may be synthesized prior to the other protein, and remains present in the expression system during synthesis of the second protein. Co-expression also refers to the insertion of nucleic acid coding for the target protein or proteins, and nucleic acid coding for the phosphatase, into an expression system, simultaneously, or at different times, wherein expression occurs simultaneously or in series.

The phosphatase may be expressed from a separate expression vector, such as, for example, but not limited to, a plasmid, virus, cosmid, bacteriophage, or a genomic integrant; from the genome; from a bacterial artificial chromosome; or from a vector that naturally expresses the phosphatase, such as, for example, a bacteriophage, in the same host cell as the DNA that encodes the target protein. The phosphatase may, for example, be expressed from the same expression vector as the target protein. The phosphatase may, for example, be expressed as part of a bicistronic message that also encodes the target protein, whereby the transcription of the message coding for the target protein and the phosphatase is under the control of the same control elements, including, for example, the same promoter. The host cell may be prokaryotic or eukaryotic. Host cells may include, for example, bacterial, yeast, insect, and mammalian cells. The expression vector may be in the form of any nucleic acid sequence capable of being expressed in a host cell. Although the type of phosphatase encoded by the expression vector is not limiting, phosphatases include, for example, protein phosphatases, including, for example, bacteriophage λ protein phosphatase (lambda-PPase). Other examples of phosphatases that may be used include shrimp alkaline phosphatase, calf intestinal phosphatase, Protein phosphatase 1, protein tyrosine phosphatase, bacterial alkaline phosphatase, T4 phosphatase, PP1 g phosphatase and PTP phosphatase. The phosphatase protein sequence may be native or may be a mutated form having phosphatase activity. Further embodiments of the invention include a method of increasing the amount of soluble protein or peptide expressed in a host cell, comprising co-expressing in a host cell a) said protein or peptide; and b) a phosphatase; wherein more soluble protein is obtained where said protein or peptide is co-expressed with said phosphatase as compared to the amount of soluble protein or peptide in the absence of said co-expression. Other embodiments also include a method of increasing the solubility of a protein or peptide expressed in a host cell, comprising a) obtaining a host cell comprising an expression vector, wherein said expression vector comprises a nucleotide sequence encoding said protein or peptide and further comprises a nucleotide sequence encoding a phosphatase, and b) growing said host cell under conditions wherein said phosphatase and said protein or peptide are expressed, wherein more soluble protein or peptide is obtained from said host cell comprising said phosphatase nucleotide sequence compared to the amount of soluble protein or peptide obtained from a host cell that does not comprise said phosphatase nucleotide sequence. Yet other embodiments include a method of increasing the solubility of a protein or peptide expressed in a host cell, comprising co-expressing a nucleotide sequence encoding a protein or peptide selected for expression in a soluble form in increased amounts with a nucleotide sequence encoding a phosphatase.

The protein or peptide may be a heterologous protein or peptide to the host cell. In some aspects, the protein or peptide may be selected from the group consisting of kinases, nuclear hormone receptor proteins, membrane-bound receptor proteins, cytokines, phosphatases, or domains or fragments thereof. In some aspects, the protein or peptide is a kinase, or a domain or fragment thereof. In some aspects, the protein or peptide is abl, or a domain or fragment thereof. In some aspects, the protein or peptide is selected from the group consisting of src, MEKK1, MASK, Mst3, and PAK4, or a domain or fragment thereof.

In some aspects, the host cell is a prokaryotic cell, for example, but not limited to, E. coli. In some aspects, the host cell is a eukaryotic cell. In some aspects, the phosphatase is encoded in the host cell DNA. In other aspects an expression vector is used to express said phosphatase. In some aspects, the expression vector comprises a nucleotide sequence that encodes said protein or peptide. In some aspects, said phosphatase and said protein or peptide are expressed from a bicistronic message. In some aspects, the expression vector is a plasmid. In some aspects, the expression vector is a phage. In some aspects, the expression vector is bacteriophage lambda. In some aspects, the expression vector is a virus. In some aspects, the phosphatase is a bacterial phosphatase. In some aspects, the phosphatase is bacteriophage lambda protein phosphatase.

### BRIEF DESCRIPTION OF THE FIGURES

FIG. 1 shows a plasmid map for an embodiment of a bacteriophage lambda protein phosphatase/target protein expression construct. Indicated on the map are a promoter, target gene, phosphatase gene, transcription terminator, a resistance gene and an origin of replication. The plasmid map is representative and is not necessarily drawn to scale.

FIG. 2 is a bar graph depicting the yield of purified soluble target protein from E. coli cells that express the target protein alone (white) and from cells that co-express a phosphatase with the target protein (black).

### DETAILED DESCRIPTION OF THE INVENTION

For purposes of the present invention, the terms “protein” and “peptide” are used interchangeably.

The protein or peptide may be homologous to the host cell, which includes, for example, proteins or peptides that are naturally encoded by the host cell, from a native DNA sequence, or a substitution, deletion, and/or insertion variant thereof. The protein or peptide may also be heterologous to the host cell, expressed on a heterologous nucleic acid sequence, which includes, for example, proteins and peptides that are not naturally expressed by the host cell, proteins and peptides that are naturally expressed or encoded by the host cell, and substitutions, deletions, and/or insertion variants of proteins and peptides that are naturally expressed or encoded by the host cell. The protein or peptide may be a fusion protein, comprising two or more polypeptides that are synthesized from a nucleic acid molecule encoding both polypeptides under the control of a single set of translational control elements. The fusion protein may also include a linker peptide situated between the polypeptides. The target protein may also be expressed as a fusion protein comprising the phosphatase.

A DNA molecule encoding the full length targeted, selected, or chosen protein of interest or fragment thereof can be prepared using well known recombinant DNA technology methods such as those set forth in Sambrook et al. (Molecular Cloning: A Laboratory Manual, Cold Spring Harbor Laboratory Press, Cold Spring Harbor, N.Y. [2001]) and/or Ausubel et al., eds, (Current Protocols in Molecular Biology, Green Publishing Assoc., Inc. John Wiley & Sons, Inc., N.Y. [1994]). A gene or cDNA encoding the protein of interest or fragment thereof may be obtained, for example, by screening a genomic or cDNA library with a suitable probe. Suitable probes include, for example, oligonucleotides, cDNA fragments, or genomic DNA fragments, that are expected to have some homology to the gene encoding the protein of interest.

If the library to be screened is an expression library, an antibody which is believed to recognize and bind an epitope of the protein of interest can be used as a screening tool. Alternatively, a gene encoding the protein of interest or fragment thereof may be prepared by chemical synthesis using methods well known to the skilled artisan such as those described by Engels et al. (Angew. Chem. Intl. Ed., 28:716-734 [1989]). These methods include, inter alia, the phosphotriester, phosphoramidite, and H-phosphonate methods for nucleic acid synthesis. A preferred method for such chemical synthesis is polymer-supported synthesis using standard phosphoramidite chemistry. Typically, the DNA encoding the protein of interest will be several hundred nucleotides in length. Nucleic acids larger than about 100 nucleotides can be synthesized as several fragments using these methods. The fragments can then be ligated together to form the full length protein of interest. Usually, the DNA fragment encoding the amino terminus of the polypeptide will have an ATG, which encodes a methionine residue. This methionine may or may not be present on the mature form of the protein of interest, depending on whether the polypeptide produced in the host cell is secreted from that cell.

In some cases, it may be desirable to prepare nucleic acid and/or amino acid variants of the naturally occurring protein of interest. Nucleic acid variants (wherein one or more nucleotides are designed to differ from the wild-type or naturally-occurring protein of interest) may be produced using site directed mutagenesis or PCR amplification where the primer(s) have the desired point mutations (see Sambrook et al., supra, and Ausubel et al., supra, for descriptions of mutagenesis techniques). Chemical synthesis using methods described by Engels et al., supra, may also be used to prepare such variants. Other methods known to the skilled artisan may be used as well. Preferred nucleic acid variants are those containing nucleotide substitutions accounting for codon preference in bacterial host cells. Other preferred variants are those encoding conservative amino acid changes. (e.g., wherein the charge or polarity of the naturally occurring amino acid side chain is not altered substantially by substitution with a different amino acid) as compared to wild type. A DNA molecule encoding a phosphatase can be prepared using the methods described above for preparation of the gene encoding the protein of interest.

Once the genes encoding the protein of interest and the phosphatase have been obtained, they may be modified using standard methods to create restriction endonuclease sites at the 5′ and/or 3′ ends. Creation of the restriction sites permits the genes to be properly inserted into amplification and/or expression vectors. Addition of restriction sites is typically accomplished using PCR, where one primer of each PCR reaction typically contains, inter alia, the nucleotide sequence of the desired restriction site.

The gene or cDNA encoding the target protein, or domain or fragment thereof can be inserted into an appropriate expression vector for expression in a host cell. The vector is selected to be functional in the particular host cell employed (i.e., the vector is compatible with the host cell machinery such that amplification and/or expression of the gene encoding the protein of interest can occur). The gene or cDNA encoding the target protein, or domain or fragment thereof may also be expressed from the genome, or from a bacterial artificial chromosome.

There are several ways to prepare the DNA construct encoding the phosphatase protein and the target protein or peptide. The phosphatase may be expressed from the same message RNA in a bicistronic construct. The phosphatase may be under the control of another promoter on the same plasmid. The phosphatase may be expressed from a separate plasmid or from the genome of the host cell. The phosphatase may also be delivered by infection with a bacteriophage that expresses the phosphatase.

Host-expression vector systems may be used to express the protein or peptide. These include, but are not limited to, microorganisms such as bacteria transformed with recombinant bacteriophage DNA, plasmid DNA or cosmid DNA expression vectors or genomic integrants of expression cassettes containing the protein or peptide coding sequence; yeast transformed with recombinant yeast expression vectors containing the protein or peptide coding sequence; insect cell systems infected with recombinant virus expression vectors (e.g., baculovirus) containing the protein or peptide coding sequence; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the protein or peptide coding sequence; or animal cell systems. The protein may also be expressed in human gene therapy systems, including, for example, expressing the protein to augment the amount of the protein in an individual, or to express an engineered therapeutic protein. The expression elements of these systems vary in their strength and specificities.

An appropriately constructed expression vector may contain: an origin of replication for autonomous replication in host cells, one or more selectable markers, a limited number of useful restriction enzyme sites, additional genes or regions that control the copy number, and active promoters. A promoter is defined as a DNA sequence that directs RNA polymerase to bind to DNA and initiate RNA synthesis, (also referred to as a “5′ flanking sequence”). A strong promoter is one that causes mRNAs to be initiated at high frequency. Expression vectors typically comprise other regulatory elements as well such as an enhancer(s), a transcriptional termination element, a ribosome binding site, a polylinker region for inserting the nucleic acid encoding the polypeptide to be expressed, and a selectable marker element. Each of these elements is discussed below. Optionally, the vector may contain a “tag” DNA sequence, i.e., an oligonucleotide sequence located at either the 5′ or 3′ end of the fusion DNA construct. The tag DNA encodes a molecule such as hexaHis, c-myc, FLAG (Invitrogen, San Diego, Calif.) or another short peptide for which antibodies and/or affinity purification resins exist. When placed in the proper reading frame, this tag will be expressed along with the fusion protein, and can serve as an affinity tag for purification of the fusion protein from the host cell and as an immunoreactive epitope useful for detection by western blotting or ELISA. Optionally, the tag can subsequently be removed from the purified fusion protein by various means such as using a specific peptidase.

The promoter may be homologous (i.e., from the same species and/or strain as the host cell), heterologous (i.e., from a species other than the host cell species or strain), hybrid (i.e., a combination of promoters from more than one source), synthetic, or it may be the native protein of interest promoter. Further, the promoter may be a constitutive or an inducible promoter. As such, the source of the promoter may be any unicellular prokaryotic or eukaryotic organism, any vertebrate or invertebrate organism, or any plant, provided that the promoter is functional in, and can be activated by, the host cell machinery.

The promoters useful in the vectors of this invention may be obtained by any of several methods well known in the art. Typically, promoters useful herein will have been previously identified by mapping and/or by restriction endonuclease digestion and can thus be isolated from the proper source using standard techniques. In some cases, the full nucleotide sequence of the promoter may be known. Here, the promoter may be synthesized using the methods described above for nucleic acid synthesis or cloning.

Where all or only a portion of the promoter sequence is known, the complete promoter may be obtained using PCR and/or by screening a genomic library with suitable oligonucleotide and/or 5′ flanking sequence fragments from the same or another species.

Suitable promoters for practicing this invention are inducible promoters such as the lux promoter, the lac promoter, the arabinose promoter, the trp promoter, the tac promoter, the tna promoter, synthetic lambda promoters (from bacteriophage lambda), and the T5 or T7 promoters. Preferred promoters include the lux, lac and arabinose promoters. These elements are often host and/or vector dependent. For example, when cloning in bacterial systems, inducible promoters such as the T7 promoter, pL of bacteriophage λ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (e.g., heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, mammalian promoters (e.g., metallothionein promoter) or mammalian viral promoters, (e.g., adenovirus late promoter; vaccinia virus 7.5K promoter; SV40 promoter; bovine papilloma virus promoter; and Epstein-Barr virus promoter) may be used.

The origin of replication element is typically a part of prokaryotic expression vectors whether purchased commercially or constructed by the user. In some cases, amplification of the vector to a certain copy number can be important for optimal expression of the protein or polypeptide of interest. In other cases, a constant copy number is preferred. In any case, a vector with an origin of replication that fulfills the requirements can be readily selected by the skilled artisan. If the vector of choice does not contain an origin of replication site, one may be chemically synthesized based on a known sequence, and ligated into the vector.

The transcription termination element is typically located 3′ of the end of the fusion protein DNA construct, and serves to terminate transcription of the RNA message coding for the fusion polypeptide. Usually, the transcription termination element in prokaryotic cells is a G-C rich fragment followed by a poly T sequence. While the element is easily cloned from a library or even purchased commercially as part of a vector, it can also be readily synthesized and introduced using methods such as those described herein.

Expression vectors typically contain a gene coding for a selectable marker. This gene encodes a protein necessary for the survival and growth of a host cell grown in a selective culture medium. Typical selection marker genes encode proteins that (a) confer resistance to antibiotics or other toxins, e.g., ampicillin, tetracycline, chloramphenicol, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, the chloramphenicol resistance gene, and the tetracycline resistance gene.

The ribosome binding element, commonly called the Shine-Dalgarno sequence in prokaryotes, is necessary for the initiation of translation of mRNA. The element is typically located 3′ to the promoter and 5′ to the coding sequence of the gene or genes to be expressed. The Shine-Dalgarno sequence is varied but is typically a polypurine (i.e., having a high A-G content). Many Shine-Dalgarno sequences have been identified, each of which can be readily synthesized using methods set forth above and used in a prokaryotic vector.

Where one or more of the elements set forth above are not already present in the vector to be used, they may be individually obtained and ligated into the vector. Methods used for obtaining each of the elements are well known to the skilled artisan and are comparable to the methods set forth above (i.e., synthesis of the DNA, library screening, and the like).

Each element may be individually ligated into the vector by cutting the vector with the appropriate restriction endonuclease(s) such that the ends of the element to be ligated in and the ends of the vector are compatible for ligation. In some cases, it may be necessary to “blunt” the ends to be ligated together in order to obtain a satisfactory ligation. Blunting can be accomplished by first filling in “sticky ends” using an enzyme such as Klenow DNA polymerase or T4 DNA polymerase in the presence of all four nucleotides. This procedure is well known in the art and is described for example in Sambrook et al., supra.

Alternatively, two or more of the elements to be inserted into the vector may first be ligated together (if they are to be positioned adjacent to each other) and then ligated into the vector.

Another method for constructing the vector is to conduct all ligations of the various elements simultaneously in one reaction mixture. Here, many nonsense or nonfunctional vectors may be generated due to improper ligation or insertion of the elements, however the functional vector may be identified by expression of the selectable marker. Proper sequence of the ligation product can be confirmed by digestion with restriction endonucleases or by DNA sequencing.

After the vector has been constructed and a fusion protein DNA construct has been inserted into the proper site of the vector, the completed vector may be inserted into a suitable host cell for fusion protein expression.

Preferred host cells suitable for the present invention are bacterial cells. For example, the various strains of E. coli (e.g., BL21, HB101, JM109, DH5α, DH10, and MC1061) are well-known host cells for use in preparing recombinant polypeptides. The choice of bacterial strain is typically made so that the strain and the expression vector to be used are compatible. Various strains of B. subtilis, Pseudomonas spp., other Bacillus spp., Streptomyces spp., and the like may also be employed in practicing this invention in conjunction with appropriate expression vectors.

Insertion (also referred to as “transformation” or “transfection”) of the vector into the selected host cell may be accomplished using such methods as calcium phosphate precipitation or electroporation. The method selected will in part be a function of the type of host cell to be used. These methods and other suitable methods are well known to the skilled artisan, and are set forth, for example, in Sambrook et al., supra.

The host cells containing the vector (i.e., transformed or transfected host cells) may be cultured using one or more standard media well known to the skilled artisan. The selected medium will typically contain all nutrients necessary for the growth and survival of the host cells. Suitable media for culturing E. coli cells, are, for example, Luria broth (“LB”), YT broth, SOB, SOC, and/or Terrific Broth.

Typically, the antibiotic or other compound useful for selective growth of the transformed cells is added as a supplement to the medium. The compound to be used will be determined by the selectable marker element present on the plasmid with which the host cell was transformed. For example, where the selectable element confers kanamycin resistance, the compound added to the culture medium will be kanamycin.

Host cells with vectors containing fusion protein DNA constructs under the control of constitutive promoters are capable of continuous fusion protein production throughout the host cell culture period. However, host cells with vectors containing fusion protein DNA constructs under the control of inducible promoters generally do not produce significant amounts of fusion protein unless the promoter is “turned on” by exposing the host cells to the proper temperature (for temperature inducible promoters) or chemical compound(s). For example, where the fusion protein DNA construct is under the control of the lac promoter, the compound IPTG (isopropyl beta.-D-thiogalactopyranoside) is typically added to the host cell culture medium to induce high-level protein production.

Identification of host cell clones that express the protein or peptide may be done by several means, including but not limited to immunological reactivity with anti-protein or peptide antibodies, and the presence of host cell-associated protein or peptide activity. Such immunological or activity assays may also be used to detect the presence of expressed phosphatase.

The solubility of the protein or peptide can be determined using standard methods known in the art. Typically, host cells are collected three to twenty hours after induction and the cells are lysed. Cell lysis may be accomplished using physical methods such as homogenization, sonication, French press, microfluidizer, or the like, or by using chemical methods such as treatment of the cells with EDTA and a detergent (see Falconer et al., Biotechnol. Bioengin. 53:453-458 [1997]) or by taking advantage of the lytic activities of some bacteriophage proteins (Crabtree, S. & Cronan, J. E., J. Bact., 1984, 158:354-356). In some cases, it may be advantageous to combine more than one technique.

Separation of soluble and insoluble material is typically accomplished by centrifugation at around 18,000×G for about 20 minutes. After the soluble and insoluble materials have been separated, visualization of soluble and insoluble fusion protein can be readily accomplished using denaturing gel electrophoresis. With this technique, equivalent volumes of soluble and insoluble fractions are applied to the gel, and the relative amount of fusion protein (or protein of interest) can be detected by staining the gel or by Western blot, provided an antibody specific for the fusion protein, the protein of interest, (depending on which entity is being assessed), or other appropriate Western blot “detection tool” is available.

Purification of the protein or peptide can be accomplished using standard techniques. If the protein or peptide has been synthesized such that it contains a tag such as Hexahistidine (“hexaHis”) or other small peptide such as myc or FLAG, for example, at either its carboxyl or amino terminus, it may be purified in a one-step process by passing the solution over an affinity column where the column matrix has a high affinity for the tag or for the polypeptide directly (i.e., an antibody specifically recognizing the protein of interest). For example, polyhistidine binds with great affinity and specificity to nickel, thus an affinity column containing nickel (such as the Qiagen nickel columns) can be used for purification of the protein of interest/hexaHis (see for example, Ausubel et al., eds., Current Protocols in Molecular Biology, Section 10.11.8, John Wiley & Sons, New York [1993]).

Where the protein or peptide has no tag and no antibodies are available, purification may be accomplished using standard methods such as those set forth below and in Marston et al. (Meth. Enz., 182:264-275 [1990]). Such procedures include, without limitation, ion exchange chromatography, hydroxylapatite chromatography, hydrophobic interaction chromatography, preparative isoelectric focusing chromatography, molecular sieve chromatography, HPLC, native gel electrophoresis in combination with gel elution, affinity chromatography, and preparative isoelectric focusing (“Isoprime” machine/technique, Hoefer Scientific). In most cases, two or more of these techniques may be combined to achieve desired purity.

### EXAMPLE 1

### Construction of a Lambda Phosphatase Co-Expression Plasmid

An open-reading frame for Aurora kinase was amplified from a Homo sapiens (human) HepG2 cDNA library (ATCC HB-8065) by the polymerase chain reaction (PCR) using the following primers:

| | |
|---|---|
| Forward primer: | TCAAAAAAGAGGCAGTGGGCTTTG |
| Reverse primer: | CTGAATTTGCTGTGATCCAGG |

The PCR product (795 base pairs expected) was gel purified as follows. The PCR product was electrophoresed on a 1% agarose gel in TAE buffer and the appropriate size band was excised from the gel and eluted using a standard gel extraction kit. The eluted DNA was ligated for 5 minutes at room temperature with topoisomerase into pSB2-TOPO. The vector pSB2-TOPO is a topoisomerase-activated, modified version of pET26b (Novagen, Madison, Wis.) wherein the following sequence has been inserted into the NdeI site: CATAATGGGCCA TCATCATCATCATCACGGTGGTCATATGTCCCTT and the following sequence inserted into the BamHI site: AAGGGGGATCCTAAACTGCAGAGATCC. The sequence of the resulting plasmid, from the Shine-Dalgarno sequence through the “original” NdeI site, the stop site and the “original” BamHI site is as follows: AAGGAGGAGATATACATAATGGGCCATCATCATCATCATCACGGTGGTCATATGTCCCTT [ORF] AAGGGGGATCCTAAACTGCAGAGATCC. The Aurora kinase expressed using this vector has 14 amino acids added to the N-terminal end (MetGlyHisHisHisHisHisHisGlyGlyHisMetSerLeu) and four amino acids added to the C-terminal end (GluGlyGlySer).

The phosphatase co-expression plasmid was then created by inserting the phosphatase gene from lambda bacteriophage into the above plasmid (Matsui T, et al., Biochem. Biophys. Res. Commun., 2001, 284:798-807). The phosphatase gene was amplified using PCR from template lambda bacteriophage DNA (HinDIII digest, New England Biolabs) using the following oligonucleotide primers:

| | |
|---|---|
| Forward primer (PPfor): | GCAGAGATCCGAATTCGAGCTCCGTC |
| | GACGGATGGAGTGAAAGAGATGCGC |
| Reverse primer (PPrev): | GGTGGTGGTGCTCGAGTGCGGCCGCA |
| | AGCTTTCATCATGCGCCTTCTCCCTG |
| | TAC |

The PCR product (744 base pairs expected) was gel purified. The purified DNA and non-co-expression plasmid DNA were then digested with SacI and XhoI restriction enzymes. Both the digested plasmid and PCR product were then gel purified and ligated together for 8 hrs at 16° C. with T4 DNA ligase and transformed into Top 10 cells using standard procedures. The presence of the phosphatase gene in the co-expression plasmid was confirmed by sequencing. For standard molecular biology protocols followed here, see also, for example, the techniques described in Sambrook et al., Molecular Cloning: A Laboratory Manual, Cold Spring Harbor Laboratory, NY, 2001, and Ausubel et al., Current Protocols in Molecular Biology, Greene Publishing Associates and Wiley Interscience, NY, 1989.

The co-expression plasmid contains both the Aurora kinase and lambda phosphatase genes under control of the lac promoter, each with its own ribosome binding site. By cloning the phosphatase into the middle of the multiple cloning site, downstream of the target gene, convenient restriction sites are available for subcloning the phosphatase into other plasmids. These sites include SacI, SalI and EcoRI between the kinase and phosphatase and HinDIII, NotI and XhoI downstream of the phosphatase.

### EXAMPLE 2

### Expression of c-Abl Protein

An open-reading frame for c-Abl was amplified from a Mus musculus (mouse) cDNA library prepared from freshly harvested mouse liver using a commercially available kit (Invitrogen) by PCR using the following primers:

| | |
|---|---|
| Forward primer: | GACAAGTGGGAAATGGAGC |
| Reverse primer: | CGCCTCGTTTCCCCAGCTC |

The PCR product (846 base pairs expected) was purified from the PCR reaction mixture using a PCR cleanup kit (Qiagen). The purified DNA was ligated for 5 minutes at room temperature with topoisomerase into pSGX3-TOPO. The vector pSGX3-TOPO is a topoisomerase-activated, modified version of pET26b (Novagen, Madison, Wis.) wherein the following sequence has been inserted into the NdeI site: CATATGTCCCTT and the following sequence inserted into the BamHI site: AAGGGCATCATCACCATCACCACTGATCC. The sequence of the resulting plasmid, from the Shine-Dalgarno sequence through the stop site and the BamHI, site is as follows: AAGGAGGA GATATACATATGTC CCTT[ORF]AAGGGCATCAT CACCATCACCACTGATCC. The c-Abl expressed using this vector had three amino acids added to its N-terminal end (Met Ser Leu) and 8 amino acids added to its C-terminal end (GluGlyHisHisHisHisHisHis).

A c-Abl/phosphatase co expression plasmid was then created by subcloning the phosphatase from the Aurora co-expression plasmid of Example 1 into the above plasmid. Both the Aurora co-expression plasmid and the Abl non-co-expression plasmid were digested 3 hrs with restriction enzymes EcoRI and NotI. The DNA fragments were gel purified and the phosphatase gene from the Aurora plasmid was ligated with the digested c-Abl plasmid for 8 hrs at 16° C. and transformed into Top 10 cells. The presence of the phosphatase gene in the resulting construct was confirmed by restriction digestion analysis.

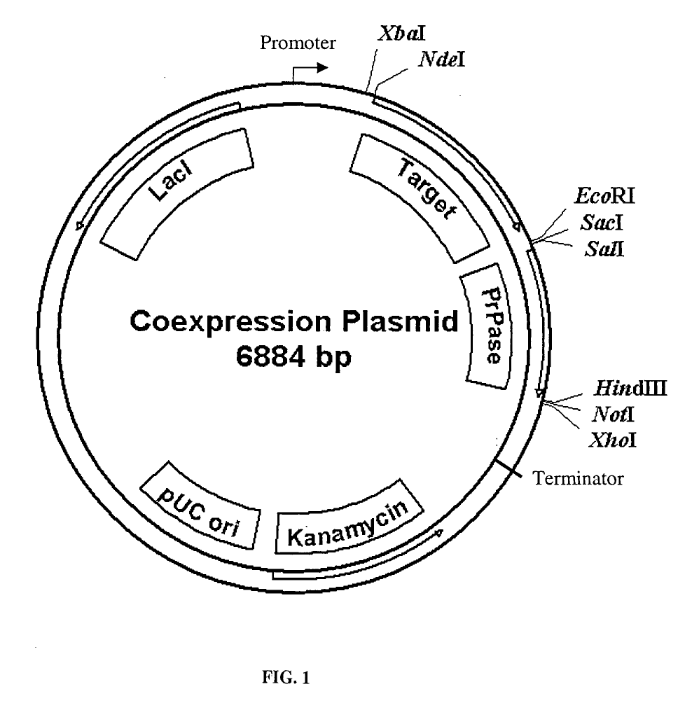

This plasmid encodes for c-Abl and lambda phosphatase co expression. It has the additional advantage of two unique restriction sites, XbaI and NdeI, upstream of the target gene that can be used for subcloning of other target proteins into this phosphatase co expressing plasmid (FIG. 1).

Protein from the non-coexpression plasmid and the phosphatase coexpression plasmids where purified as follows. The non-co-expression plasmid was transformed into chemically competent BL21(DE3)Codon+RIL (Stratagene) cells and the co-expression plasmid was transformed into BL21(DE3) pSA0145 (a strain that expresses the lytic genes of lambda phage and lyses upon freezing and thawing (Crabtree S, Cronan J E Jr. J Bacteriol 1984 April;158(1):354-6)) and plated onto petri dishes containing LB agar with kanamycin. Isolated, single colonies were grown to mid-log phase and stored at −80° C. in LB containing 15% glycerol. This glycerol stock was streaked on LB agar plates with kanamycin and a single colony was used to inoculate 10 ml cultures of LB with kanamycin and chloramphenicol, which was incubated at 30° C. overnight with shaking. This culture was used to inoculate a 2 L flask containing 500 mls of LB with kanamycin and chloramphenicol, which was grown to mid-log phase at 37° C. and induced by the addition of IPTG to 0.5 mM final concentration. After induction flasks were incubated at 21° C. for 18 hrs with shaking.

Cells were collected by centrifugation. Cultures of BL21(DE3) pSA0145 were lysed by a freeze thaw cycle. Cultures of BL21 (DE3)Codon+RIL cells were lysed in lysis buffer (50 mM Tris HCl (pH 7.5), 20 mM imidazole, 0.1% v/v Tween-20) by sonication. Lysed cultures were centrifuged to remove cell debris. The soluble fraction was purified over an IMAC column charged with nickel (Pharmacia, Uppsala, Sweden), and eluted under native conditions with a linear gradient of imidazole. Protein from the non-phosphatase vector was further purified using a Superdex 200 gel filtration column and then a MonoQ column. Protein from the phosphatase vector was further purified using a Superdex 200 gel filtration column. Fractions containing the purified protein were pooled and concentrated in centricon concentrators and protein yields determined by measuring an optical density at 280 nm and using a calculated extinction coefficient.

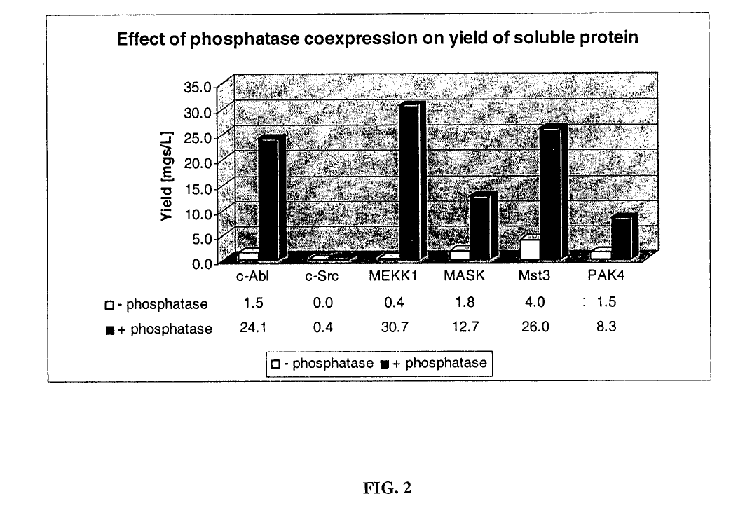

Expression from the original non-co-expression plasmid yielded 3 mgs of soluble protein from 2 L, or 1.5 mgs/L. Expression of the phosphatase co expression plasmid yielded 96 mgs from 4 L, or 24 mgs/L (FIG. 2).

### EXAMPLE 3

### Expression of c-Src Protein

An open-reading frame c-Src was amplified from Homo sapiens (human) HepG2 genomic DNA (ATCC HB-80675) by PCR with the following primers:

| | |
|---|---|
| Forward primer: | GGCCTGGCCAAGGATGC |
| Reverse primer: | CCCCGGGCTGGTACTGGGGCTC |

The PCR product (831 base pairs expected) was gel purified and ligated for 5 minutes at room temperature with topoisomerase into pSGX2-TOPO. The vector pSGX2-TOPO is a topoisomerase activated, modified version of pET26b (Novagen, Madison, Wis.) wherein the following sequence has been inserted into the NdeI site: CATATGGCTCATCATCACCATCACCACTCCCTT and the following sequence has been inserted into the BamHI site:AAGGGTGATCC. The sequence of the resulting plasmid, from the Shine-Dalgarno sequence through the NdeI site, the stop site and the BamHI site is as follows: AAGGAGGAGATATACATATGG CTCATCATCACCATCACCACTCCCTT [ORF] AAGGGTGATCC. The c-Src expressed using this vector has 10 amino acids added to the N-terminal end (MetAlaHisHisHisHisHisHisSerLeu) and two amino acids added to the C-terminal end (GluGly).

A c-Src/phosphatase co expression plasmid was then created by subcloning the c-Src gene from the above plasmid into the Abl co-expression plasmid. The c-Src gene, including some flanking plasmid sequence, was amplified from the above plasmid by PCR using the following primers:

| | |
|---|---|
| Forward primer (T7): | TAATACGACTCACTATAGGG |
| Reverse primer (pSB21): | TGTCGACGGAGCTCGAATTC |

After gel purification of the PCR product, both the Abl co-expression plasmid and the PCR product were digested 3 hrs with restriction enzymes XbaI and EcoRI. The appropriately sized fragments were gel purified and the fragment encoding the c-Src gene from the Src non-co-expression plasmid was ligated with the digested Abl co-expression plasmid for 8 hrs at 16° C. and transformed into Top10 cells. The correct construct was confirmed by DNA sequencing.

Small scale expression and purification experiments were performed for both the Src co-expression and the non-co-expression plasmids. Plasmids were transformed into chemically competent BL21(DE3)Codon+RIL (Stratagene) cells and plated onto petri dishes containing LB agar with 30 μg/ml of kanamycin. Small (1.5 ml) cultures were grown from isolated, single colonies to mid-log phase and 100 μl stored at −80° C. in LB containing 15% glycerol. The remaining culture was induced by the addition of IPTG to a final concentration of 1 mM and incubated at 25° C. for 12 hrs. Cells were recovered by centrifugation and resuspended in lysis buffer and lysed by sonication. After centrifugation to remove insoluble material, soluble proteins were captured using Ni-NTA magnetic beads (Qiagen) and visualized by SDS-PAGE. Gels showed that without phosphatase co expression, c-Src was insoluble, whereas with phosphatase co-expression some c-Src was soluble.

A large scale preparation of c-Src from the phosphatase coexpression plasmid was therefore done. This glycerol stock of the Src co-expression plasmid in BL21(DE3)Codon+RIL cells was streaked on LB agar with kanamycin and a single colony was used to inoculate 10 mls of LB with kanamycin and chloramphenicol, which was incubated at 30° C. overnight, shaking at 250 rpm. This culture was used to inoculate flasks 500 ml of LB with kanamycin and chloramphenical, which were grown to mid-log phase at 37° C. and induced by the addition of IPTG to 0.5 mM final concentration. After induction flasks were incubated at 22° C. for 18 hrs with shaking.

Cells were collected by centrifugation, lysed by sonication and centrifuged to remove cell debris. The soluble fraction was purified over an IMAC column charged with nickel (Pharmacia, Uppsala, Sweden), and eluted under native conditions with a linear gradient of imidazole. Protein was further purified using using a desalting column, followed by a Resource Q anion exchange column. The protein was eluted with a 0-50% gradient. Finally, the protein was subjected to a gel filtration column (Superdex 200). The final yield of soluble protein from the phosphatase vector was determined to be 1.3 mgs from 3 L, or 0.4 mgs/L. As stated, the protein from the non-phosphatase vector was found to be completely insoluble.

### EXAMPLE 4

### Expression of MEKK1 Protein

An open-reading frame for MEKK1 was amplified from a Sus scrofa (pig) cDNA library (ATCC CCL-33) by PCR using the following primers:

| | |
|---|---|
| Forward primer: | GCTTCAACTACAACCTGCAC |
| Reverse primer: | CTGCTGAAAAATTCCTCGTAGC |

The PCR product (945 base pairs expected) was gel purified and ligated into pSGX3-TOPO as described in example 2 for the c-Abl protein. The MEKK1 gene in the resulting plasmid was verified by sequencing.

A MEKK1/phosphatase co expression plasmid was then created by subcloning the MEKK1 gene from the above plasmid into the Abl co-expression plasmid. The MEKK1 gene, including some flanking plasmid sequence, was amplified from the above plasmid by PCR using the following primers:

| | |
|---|---|
| Forward primer (T7): | TAATACGACTCACTATAGGG |
| Reverse primer (pSB21): | TGTCGACGGAGCTCGAATTC |

After gel extraction of purification of the PCR product, both the Abl co-expression plasmid and the PCR product were digested 3 hrs with restriction enzymes NdeI and EcoRI. The fragments were gel purified and that encoding the MEKK1 gene from the MEKK1 non-co-expression plasmid was ligated with the digested Abl co-expression plasmid for 8 hrs at 16° C. and transformed into Top10 cells. The correct construct was confirmed by DNA sequencing.

Plasmids were transformed into chemically competent BL21(DE3) Codon+RIL (Stratagene) cells and plated onto petri dishes containing LB agar with 30 μg/ml of kanamycin. Isolated, single colonies were grown to mid-log phase and stored at −80° C. in LB containing 15% glycerol. This glycerol stock was streaked on LB agar with kanamycin and a single colony was used to inoculate 10 mls of LB with kanamycin and chloramphenicol, which was incubated at 30° C. overnight, shaking at 250 rpm. This culture was used to inoculate flasks containing 500 ml of LB with kanamycin and chloramphenicol, which were grown to mid-log phase at 37° C. and induced by the addition of IPTG to 0.5 mM final concentration. After induction flasks were incubated at 22° C. for 18 hrs with shaking.

Cells were collected by centrifugation, lysed lysed by sonication and centrifuged to remove cell debris. The soluble fraction was purified over an IMAC column charged with nickel (Pharmacia, Uppsala, Sweden), and eluted under native conditions with a linear gradient of imidazole. At this point the protein from the non-phosphatase vector was buffer exchanged using a desalting column and further purified using an anion exchange column. Both proteins proteins were further purified using a Superdex 200 gel filtration column. From the non phosphatase vector 1.4 mgs was recovered from 4 L, or 0.4 mgs/L. From the phosphatase co expression vector 92 mgs was purified from 3 L, or 31 mgs/L. (FIG. 2).

### EXAMPLE 5

### Expression of MASK Protein

An open-reading frame for MASK was amplified from a Drosophila melanogaster (fruit fly) cDNA library (ATCC CRL-1963) by PCR using the following primers:

| | |
|---|---|
| Forward primer: | TCCTGGACGGAGAAAGTC |
| Reverse primer: | CTTCGTCGCCCTTGGAGAC |

The PCR product (864 base pairs expected) was gel purified and ligated into pSGX2-TOPO as described in example 3 for the c-Src protein. The MASK gene in the resulting plasmid was verified by sequencing.

A MASK/phosphatase co expression plasmid was then created by subcloning the MASK gene from the above plasmid into the Abl co-expression plasmid. The MASK gene, including some flanking plasmid sequence, was amplified from the above plasmid by PCR using the primers:

| | |
|---|---|
| Forward primer (T7): | TAATACGACTCACTATAGGG |
| Reverse primer (pSB21): | TGTCGACGGAGCTCGAATTC |

After gel extraction of purification of the PCR product, both the Abl co-expression plasmid and the PCR product were digested 3 hrs with restriction enzymes XbaI and EcoRI. The digestion reactions were gel purified. The fragment encoding the MASK gene from the MASK non-co-expression plasmid was ligated with the digested Abl co-expression plasmid for 8 hrs at 16° C. and transformed into Top 10 cells. The correct construct was confirmed by DNA sequencing.

Plasmids were transformed into chemically competent BL21(DE3)Codon+RIL (Stratagene) cells and plated onto petri dishes containing LB agar with 30 μg/ml of kanamycin. Isolated, single colonies were grown to mid-log phase and stored at −80° C. in LB containing 15% glycerol. This glycerol stock was streaked on LB agar with kanamycin and a single colony was used to inoculate 5 mls of LB with kanamycin and chloramphenicol, which was incubated at 30° C. overnight, shaking at 250 rpm. This culture was used to inoculate flasks containing 500 ml of LB with kanamycin, which were grown to mid-log phase at 37° C. and induced by the addition of IPTG to 0.5 mM final concentration. After induction flasks were incubated at 22° C. for 18 hrs with shaking.

Cells were collected by centrifugation, lysed by sonication and centrifuged to remove cell debris. The soluble fraction was purified over an IMAC column charged with nickel (Pharmacia, Uppsala, Sweden), and eluted under native conditions with a linear gradient of imidazole. At this point the protein from the phosphatase vector was further purified using a Superdex 200 column then buffer exchanged using a desalting column and further purified using a Resource Q anion exchange column. The protein from the non-phosphatase vectors was further purified by gel filtration using a Superdex 200. From the non phosphatase vector 5.5 mgs was recovered from 3 L, or 1.8 mgs/L. From the phosphatase co expression vector 38 mgs was purified from 3 L, or 13 mgs/L.

### EXAMPLE 6

### Expression of Mst3 Protein

An open-reading frame for Mst3 was amplified from Homo sapiens (human) HepG2 genomic DNA (ATCC HB-80675) PCR using the following primers:

| | |
|---|---|
| Forward primer: | GACCCAGAAGAGCTTTTTACAAAAC |
| Reverse primer: | CTTCCGCGTCGGAATCCTC |

The PCR product (879 base pairs expected) was gel purified and ligated into pSGX3-TOPO as described in example 2 for the c-Abl protein. The Mst3 gene in the resulting plasmid was verified by sequencing.

A Mst3/phosphatase co expression plasmid was then created by subcloning the Mst3 gene from the above plasmid into the Abl co-expression plasmid. The Mst3 gene, including some flanking plasmid sequence, was amplified from the above plasmid PCR with the following primers:

| | |
|---|---|
| Forward primer (T7): | TAATACGACTCACTATAGGG |
| Reverse primer (pSB21): | TGTCGACGGAGCTCGAATTC |

After gel purification of the PCR product, both the Abl co-expression plasmid and the PCR product were digested 3 hrs with restriction enzymes XbaI and EcoRI. The digestion reactions were gel purified. The fragment encoding the Mst3 gene from the Mst3 non-co-expression plasmid was ligated with the digested Abl co-expression plasmid for 8 hrs at 16° C. and transformed into Top10 cells. The correct construct was confirmed by DNA sequencing.

Plasmids were transformed into chemically competent BL21 (DE3)Codon+RIL (Stratagene) cells and plated onto petri dishes containing LB agar with 30 μg/ml of kanamycin. Isolated, single colonies were grown to mid-log phase and stored at −80° CC in LB containing 15% glycerol. This glycerol stock was streaked on LB agar with kanamycin and a single colony was used to inoculate 10 mls of LB with kanamycin and chloramphenicol, which was incubated at 30° C. overnight, shaking at 250 rpm. This culture was used to inoculate flasks containing 500 ml of LB with kanamycin and chloramphenicaol, which were grown to mid-log phase at 37° C. and induced by the addition of IPTG to 0.5 mM final concentration. After induction flasks were incubated at 22° C. for 18 hrs hrs with shaking.

Cells were collected by centrifugation, lysed by sonication and centrifuged to remove cell debris. The soluble fraction was purified over an IMAC column charged with nickel (Pharmacia, Uppsala, Sweden), and eluted under native conditions with a linear gradient of imidazole. At this point the protein from the non-phosphatase vector was further purified using a MonoQ anion exchange column. Both the protein from the phosphatase and non-phosphatase vectors were then further purified by gel filtration using a Superdex 200 column. From the non phosphatase vector 2.6 mgs was recovered from 4 L, or 0.7 mgs/L. From the phosphatase co expression vector 78 mgs was purified from 3 L, or 26 mgs/L. (FIG. 2).

### EXAMPLE 7

### Expression of Pak4 Protein

An open-reading frame for PAK4 was amplified from a Homo sapiens (human) HepG2 cDNA library (ATCC HB-8065) by PCR using the primers:

| | |
|---|---|
| Forward primer: | TCCCATGAGCAGTTCCGGGCTG |
| Reverse primer: | CTCTGGTGCGGTTCTGGCGCATG |

The PCR product (876 base pairs expected) was purified by gel extraction and ligated into pSGX2-TOPO as described in example 3 for the c-Src protein. The PAK4 gene in the resulting plasmid was verified by sequencing.

A PAK4/phosphatase co expression plasmid was then created by subcloning the PAK4 gene from the above plasmid into the Abl co-expression plasmid. The PAK4 gene, including some flanking plasmid sequence, was amplified from the above plasmid by PCR using the following primers:

| | |
|---|---|
| Forward primer (T7): | TAATACGACTCACTATAGGG |
| Reverse primer (pSB21): | TGTCGACGGAGCTCGAATTC |

After gel extraction of purification of the PCR product, both the Abl co-expression plasmid and the PCR product were digested 3 hrs with restriction enzymes XbaI and EcoRI. The digestion reactions were gel purified. The fragment encoding the PAK4 gene from 5599a1BNt1p1 was ligated with the digested Abl co-expression plasmid for 8 hrs at 16° C. and transformed into Top10 cells. The correct construct was confirmed by DNA sequencing.

Plasmids were transformed into chemically competent BL21(DE3)Codon+RIL (Stratagene) cells and plated onto petri dishes containing LB agar with 30 μg/ml of kanamycin. Isolated, single colonies were grown to mid-log phase and stored at −80° CC in LB containing 15% glycerol. This glycerol stock was streaked on LB agar with kanamycin and a single colony was used to inoculate 10 mls of LB with kanamycin and chloramphenicol, which was incubated at 37° C. overnight, shaking at 250 rpm. This culture was used to inoculate flasks containing 500 ml of LB with kanamycin and chloramphenicol, which were grown to mid-log phase at 30° C. and induced by the addition of IPTG to 0.5 mM final concentration. After induction flasks were incubated at 22° C. for 18 hrs with shaking.

Cells were collected by centrifugation, lysed by sonication and centrifuged to remove cell debris. The soluble fraction was purified over an IMAC column charged with nickel (Pharmacia, Uppsala, Sweden), and eluted under native conditions with a linear gradient of imidazole. At this point the protein from the non-phosphatase vector was buffer exchanged using a desalting column and then purified using a cation exchange column. Both the protein from the phosphatase and non-phosphatase vectors were then further purified by gel filtration using a Superdex 200 column. From the phosphatase coexpression vector 50 mgs was purified from 6 L, or 5 mgs/L. (FIG. 2).

### EXAMPLE 8

### Enhancing Solubility of a Protein Co-Expressed from the Same Plasmid

The phosphatase and the target protein may be co-expressed from separate messages. This is achieved, for example, by inserting the phosphatase gene into a region of the plasmid that is under the control of a separate promoter from the target gene promoter. Or by introducing a separate promoter between the target gene and the phosphatase gene in the described constructs such that the ribosome binding site (RBS) of the phosphatase is appropriately placed to put the phosphatase under the control of this second promoter. The resulting plasmid has two sections: one corresponding to promoter-RBS-target protein and one corresponding to promoter-RBS-phosphatase. The two promoters may respond to the same inducing agent or different inducing agents to achieve separate control.

### EXAMPLE 9

### Enhancing Solubility of a Protein Expressed from a Separate Plasmid

The phosphatase and the target protein may be expressed from separate plasmids. This is achieved, for example, by inserting the phosphatase gene into a plasmid different from, but compatible with, the plasmid coding for the target protein, under the control of a promoter. It is known in the art which plasmids are compatible with each other for simultaneous propagation. The promoter used may be the same promoter that controls the expression of the target gene, in which case expression of both the target gene and phosphatase would be induced by the same agent. Alternatively, it may be under control of a different promoter, in which case separate control of phosphatase induction times and induction strengths is achieved.

The present invention is not to be limited in scope by the exemplified embodiments, which are intended as illustrations of single aspects of the invention. Indeed, various modifications of the invention in addition to those described herein will become apparent to those having skill in the art from the foregoing description and accompanying drawings. Such modifications are intended to fall within the scope of the invention. References cited throughout this application are examples of the level of skill in the art and are hereby incorporated by reference herein in their entirety.

1. A method of increasing the amount of soluble protein or peptide expressed in a host cell, comprising co-expressing in a host cell
a) said protein or peptide; and
b) a phosphatase;
wherein more soluble protein is obtained where said protein or peptide is co-expressed with said phosphatase as compared to the amount of soluble protein or peptide in the absence of said co-expression.

2. The method of claim 1, wherein the protein or peptide is a heterologous protein or peptide to the host cell.

3. The method of claim 1, wherein the protein or peptide is selected from the group consisting of kinases, nuclear hormone receptor proteins, membrane-bound receptor proteins, cytokines, phosphatases, or domains or fragments thereof.

4. The method of claim 1, wherein the protein or peptide is a kinase, or a domain or fragment thereof.

5. The method of claim 1, wherein the protein or peptide is abl, or a domain or fragment thereof.

6. The method of claim 1, wherein the protein or peptide is selected from the group consisting of src, MEKK1, MASK, Mst3, and PAK4, or a domain or fragment thereof.

7. The method of claim 1, wherein the protein or peptide is a kinase, or a domain or fragment thereof.

8. The method of claim 1, wherein the host cell is a prokaryotic cell.

9. The method of claim 7, wherein the host cell is E. coli.

10. The method of claim 1, wherein the phosphatase is encoded in the host cell DNA.

11. The method of claim 1, wherein an expression vector is used to express said phosphatase.

12. The method of claim 10, wherein the expression vector comprises a nucleotide sequence that encodes said protein or peptide.

13. The method of claim 10, wherein said phosphatase and said protein or peptide are expressed from a bicistronic message.

14. The method of claim 10, wherein said expression vector is a plasmid.

15. The method of claim 10, wherein said expression vector is a phage.

16. The method of claim 14, wherein said expression vector is bacteriophage lambda.

17. The method of claim 10, wherein said expression vector is a virus.

18. The method of claim 1, wherein said phosphatase is a bacterial phosphatase.

19. The method of claim 1, wherein said phosphatase is bacteriophage lambda protein phosphatase.

20. A method of increasing the solubility of a protein or peptide expressed in a host cell, comprising
a. obtaining a host cell comprising an expression vector, wherein said expression vector comprises a nucleotide sequence encoding said protein or peptide and further comprises a nucleotide sequence encoding a phosphatase, and
b. growing said host cell under conditions wherein said phosphatase and said protein or peptide are expressed,
wherein more soluble protein or peptide is obtained from said host cell comprising said phosphatase nucleotide sequence compared to the amount of soluble protein or peptide obtained from a host cell that does not comprise said phosphatase nucleotide sequence.

21. The method of claim 19, wherein the protein or peptide is selected from the group consisting of kinases, nuclear hormone receptor proteins, membrane-bound receptor proteins, cytokines, phosphatases, or domains or fragments thereof.

22. The method of claim 19, wherein the protein or peptide is a kinase, or a domain or fragment thereof.

23. The method of claim 19, wherein the protein or peptide is abl, or a domain or fragment thereof.

24. The method of claim 19, wherein the protein or peptide is selected from the group consisting of Src, MEKK1, MASK, Mst3, and PAK4, or a domain or fragment thereof.

25. The method of claim 19, wherein the host cell is a prokaryotic cell.

26. The method of claim 19, wherein the host cell is E. coli.

27. The method of claim 19, wherein the phosphatase is encoded in the host cell DNA.

28. The method of claim 19, wherein an expression vector is used to express said phosphatase.

29. The method of claim 19, wherein said phosphatase and said protein or peptide are expressed from a bicistronic message.

30. The method of claim 26, wherein said expression vector is a plasmid.

31. The method of claim 26, wherein said expression vector is a phage.

32. The method of claim 26, wherein said expression vector is bacteriophage lambda.

33. The method of claim 26, wherein said expression vector is a virus.

34. The method of claim 26, wherein said phosphatase is a bacterial phosphatase.

35. The method of claim 26, wherein said phosphatase is bacteriophage lambda protein phosphatase.

36. A method of increasing the solubility of a protein or peptide expressed in a host cell, comprising co-expressing a nucleotide sequence encoding a protein or peptide selected for expression in a soluble form in increased amounts with a nucleotide sequence encoding a phosphatase.

37. The method of claim 36, wherein the protein or peptide is selected from the group consisting of kinases, nuclear hormone receptor proteins, membrane-bound receptor proteins, cytokines, phosphatases, or domains or fragments thereof.

38. The method of claim 36, wherein the protein or peptide is a kinase, or a domain or fragment thereof.

39. The method of claim 36, wherein the protein or peptide is abl, or a domain or fragment thereof.

40. The method of claim 36, wherein the protein or peptide is selected from the group consisting of Src, MEKK1, MASK, Mst3, and PAK4, or a domain or fragment thereof.

41. The method of claim 36, wherein the host cell is a prokaryotic cell.

42. The method of claim 36, wherein the host cell is E. coli.

43. The method of claim 36, wherein the phosphatase is encoded in the host cell DNA.

44. The method of claim 36, wherein an expression vector is used to express said phosphatase.

45. The method of claim 43, wherein said expression vector is a plasmid.

46. The method of claim 43, wherein said expression vector is a phage.

47. The method of claim 43, wherein said expression vector is bacteriophage lambda.

48. The method of claim 43, wherein said expression vector is a virus.

49. The method of claim 43, wherein said phosphatase is a bacterial phosphatase.

50. The method of claim 43, wherein said phosphatase is bacteriophage lambda protein phosphatase.
